(19) United States
(12) Patent Application Publication
(10) Pub. No.: US 2016/0081627 A1
(43) Pub. Date: Mar. 24, 2016

(54) SYSTEM METHOD FOR ASSESSING FITNESS STATE VIA A MOBILE DEVICE

(71) Applicant: QUALCOMM Incorporated, San Diego, CA (US)

(72) Inventors: Justin Patrick McGloin, Los Altos, CA (US); Russel Allyn Martin, Menlo Park, CA (US); Radu Pitigoi-Aron, San Jose,, CA (US); Ramin Samadani, Menlo Park, CA (US); Igor Tchertkov, San Jose, CA (US)

(21) Appl. No.: 14/494,350

(22) Filed: Sep. 23, 2014

(51) Int. Cl. A61B 5/00; A61B 5/0402; A61B 5/0205; A61B 5/11; A61B 5/0488; A61B 5/0295

(52) U.S. Cl. CPC A61B 5/7264; A61B 5/0488; A61B 5/0295; A61B 5/0205; A61B 5/1118; A61B 5/681; A61B 5/742; A61B 5/749; A61B 5/4866; A61B 5/0402; A61B 5/7405; A61B 5/0015; A61B 5/02416

(57) ABSTRACT

Methods, systems, computer-readable media, and apparatuses for assessing a fitness state of a user via a mobile device are presented. In some implementations, a first physiological measurement of the user during a first level of a physical activity is obtained via one or more sensors. A second physiological measurement during a second level of the physical activity is obtained via the one or more sensors. A transient physiological measurement based on the first physiological measurement and the second physiological measurement is determined. The physical activity is classified based on one or more motion measurements obtained via the one or more sensors. A fitness profile indicative of a fitness state of the user is generated based at least in part on the determined transient physiological measurement and the classified physical activity.

### BACKGROUND

Aspects of the disclosure relate to mobile devices, and more particularly, a system and method for obtaining at least one bodily function measurement of a user operating a mobile device.

It is often desirable for a user to be aware of his/her fitness state. Small and lightweight devices are increasingly being used in many applications related to monitoring health and fitness of individuals. Many of these wearable devices can be used to assess the fitness state of an individual. One well-known metric for assessing the fitness state of an individual is heart rate recovery (HRR) of the individual after seizing a physical activity. Moreover, numerous recent studies have confirmed heart rate recovery after exercise as a powerful predictor of mortality from heart disease. Another recent study found that the rate of ramping up the heart rate (HR) in the beginning of the physical activity could be a complementary predictor of the cardiovascular condition of the person. However, to the extent that existing devices can measure HRR, they only do so by calculating the difference in heart rate of an individual between an intense physical activity and sitting still. Additionally, many of the existing solutions exist in a clinical setting and cannot correlate the measured HRR to a real-world physical activity performed by the individual.

Accordingly, a need exists for a small and lightweight device that is able to measure HRR between a first real-world physical activity level and a second real-world physical activity level, where the second real-world activity level is not necessarily sitting still or resting.

### BRIEF SUMMARY

Certain implementations are described that assess a fitness state of a user via a mobile device.

In some implementations, a method for assessing a fitness state of a user via a mobile device includes obtaining, via one or more sensors, a first physiological measurement of the user during a first level of a physical activity. The method also includes obtaining, via the one or more sensors, a second physiological measurement during a second level of the physical activity. The method additionally includes determining, via a processor of the mobile device, a transient physiological measurement based on the first physiological measurement and the second physiological measurement. The method further includes classifying the physical activity based on one or more motion measurements obtained via the one or more sensors. Moreover, the method includes generating a fitness profile indicative of a fitness state of the user based at least in part on the determined transient physiological measurement and the classified physical activity.

In some implementations, the first physiological measurement and the second physiological measurement comprises at least one of a heart rate measurement, an electromyography response measurement, or a blood pressure measurement.

In some implementations, the determined transient physiological measurement comprises a heart rate recovery (HRR) measurement.

In some implementations, the one or more sensors comprises at least an accelerometer.

In some implementations, the method also includes monitoring the determined transient physiological measurement and subsequently determined transient physiological measurements over a period of time.

In some implementations, generating the fitness profile is further based at least in part on subsequently classified physical activities and the subsequently determined transient physiological measurements.

In some implementations, generating the fitness profile is further based at least in part on a frequency of the user to engage in the classified physical activity.

In some implementations, the method also includes presenting, via a display of the mobile device, the fitness profile to the user within a graphical user interface (GUI).

In some implementations, classifying the physical activity includes categorizing the physical activity into one or more predefined physical activity categories.

In some implementations, the first level of physical activity and the second level of the physical activity are above a level of physical activity indicative of the user being in a resting state.

In some implementations, the first physiological measurement and the second physiological measurement are opportunistically obtained by the mobile device.

In some implementations, a mobile device for assessing a fitness state of a user includes an outer body sized to be portable for a user, a processor contained within the outer body, and a plurality of sensors coupled to the outer body for obtaining data accessible by the processor. The one or more of the sensors is configured to obtain a first physiological measurement of the user during a first level of physical activity. The one or more sensors is also configured to obtain a second physiological measurement of the user during a second level of physical activity. The processor is configured to determine a transient physiological measurement based on the first physiological measurement and the second physiological measurement. The processor is also configured to classify the physical activity based on one or more motion measurements obtained via the one or more sensors. The processor is additionally configured to generate a fitness profile indicative of a fitness state of the user based at least in part on the determined transient physiological measurement and the classified physical activity.

In some implementations, an apparatus for assessing a fitness state of a user includes means for obtaining, via one or more sensors, a first physiological measurement of a user during a first level of a physical activity. The apparatus also includes means for obtaining a second physiological measurement during a second level of the physical activity. The apparatus additionally includes means for determining, via the one or more sensors, a transient physiological measurement based on the first physiological measurement and the second physiological measurement. The apparatus further includes means for classifying the physical activity based on one or more motion measurements obtained via the one or more sensors. The apparatus also includes means for generating a fitness profile indicative of a fitness state of the user based at least in part on the determined transient physiological measurement and the classified physical activity.

In some implementations, one or more non-transitory computer-readable media storing computer-executable instructions for assessing a fitness state of a user, when executed, cause one or more computing devices included in a mobile device to obtain, via one or more sensors, a first physiological measurement of the user during a first level of a physical activity. The computer-executable instructions, when executed, also cause the one or more computing devices to obtain, via the one or more sensors, a second physiological measurement during a second level of the physical activity. The computer-executable instructions, when executed, also cause the one or more computing devices to determine, via a processor of the mobile device, a transient physiological measurement based on the first physiological measurement and the second physiological measurement. The computer-executable instructions, when executed, also cause the one or more computing devices to classify the physical activity based on one or more motion measurements obtained via the one or more sensors. The computer-executable instructions, when executed, also cause the one or more computing devices to generate a fitness profile indicative of a fitness state of the user based at least in part on the determined transient physiological measurement and the classified physical activity.

### BRIEF DESCRIPTION OF THE DRAWINGS

Aspects of the disclosure are illustrated by way of example. In the accompanying figures, like reference numbers indicate similar elements.

FIG. 1 is a block diagram of an exemplary mobile device, according to some implementations;

FIG. 2 illustrates a mobile device configured to obtain bodily function measurements of a user, according to some implementations;

FIG. 3 is a flow diagram of a process for determining a heart rate recovery (HRR) measurement of a user, according to some implementations;

FIG. 4 is a flow diagram of a process for classifying a physical activity, according to some implementations;

FIG. 5 is a flow diagram of a process for generating a fitness profile for a user, according to some implementations;

FIG. 6A illustrates an exemplary absolute fitness profile displayed on a mobile device;

FIG. 6B illustrates an exemplary relative fitness profile displayed on a mobile device

FIG. 6C illustrates an exemplary performance profile displayed on a mobile device; and

FIG. 7 illustrates an example of a computing system in which one or more embodiments may be implemented.

### DETAILED DESCRIPTION

Several illustrative embodiments will now be described with respect to the accompanying drawings, which form a part hereof. While particular embodiments, in which one or more aspects of the disclosure may be implemented, are described below, other embodiments may be used and various modifications may be made without departing from the scope of the disclosure or the spirit of the appended claims.

Implementations disclosed herein pertain to an ongoing and automatic method for measuring and tracking physiological transient measurements and using them as an indicator of a user's fitness state. The physiological transient measurements can include a heart rate recovery (HRR) measurement (e.g., the speed at which a user's heart rate (HR) returns to normal after exercise). The measurements may not require user intervention and the actions for assessing the fitness state may be taken programmatically and algorithmically. That is, the measurements may be obtained opportunistically during the course of the user's “regular” daily activities. The measurement may be performed by a small and lightweight mobile device such as a wristwatch, or by a combination of mobile devices such as a wristwatch linked to a smartphone. The mobile device(s) may also include an accelerometer, computational core (such as a microcontroller or DSP), algorithms such as motion/activity classifiers, pedometer, etc. The obtained measurements may be stored in memory within the mobile device(s).

Additionally, implementations disclosed herein can classify a physical activity (motion activity) based on measurements obtained via one or more sensors (e.g., an accelerometer or gyroscope). The physical activities can be classified by type (e.g., walking, running, cycling, stair-climbing, etc.)

The measurement results can include parameters of ramping up the HR and physical activity, as well as attenuating HR down to the rest level or reduction of physical activity. The aforementioned parameters can include rising and falling time, rate of rising and falling HR and physical activity, level and duration of elevated HR and physical activity, degree of correlation between HR and the classified physical activity (motion activity), etc. This opportunistic and ongoing data collection can be repeated for many days, weeks, months etc. Implementations described herein may analyze the duration and strenuous level of the classified physical (motion) activity, HR profile during the classified physical activity and HR transients (e.g., HRR measurement) in the beginning and after the classified physical activity, etc. Over the weeks and perhaps months of data collection and data analysis, the data could reveal state of the user's fitness and its evolution over the time. This data can be presented to the user in the form of a fitness profile indicative of the user's fitness state. The fitness profile can include the user's transient physiological measurement data for each classified physical activity over a specified time period. A corresponding analysis as the user's fitness state based on the aforementioned information may also be provided to the user.

The fitness state can include an absolute fitness state or a relative fitness state. The absolute fitness state can provide an overall “picture” of the user's fitness and may be dependent on the user's age and gender. The relative fitness state can provide the user with a fitness indication relative to the user's fitness state in the past. As such, the user may “compete” against himself/herself to improve his/her fitness state as time goes on. The fitness metric can capture a change of state of the user's fitness over time.

In some embodiments, heart rate (HR) can be measured by several methods, EKG (aka ECG), Photoplethysmogram (PPG), Impedance PG (IPG). It can be appreciated that all these methods and others can be applicable to the embodiments described herein.

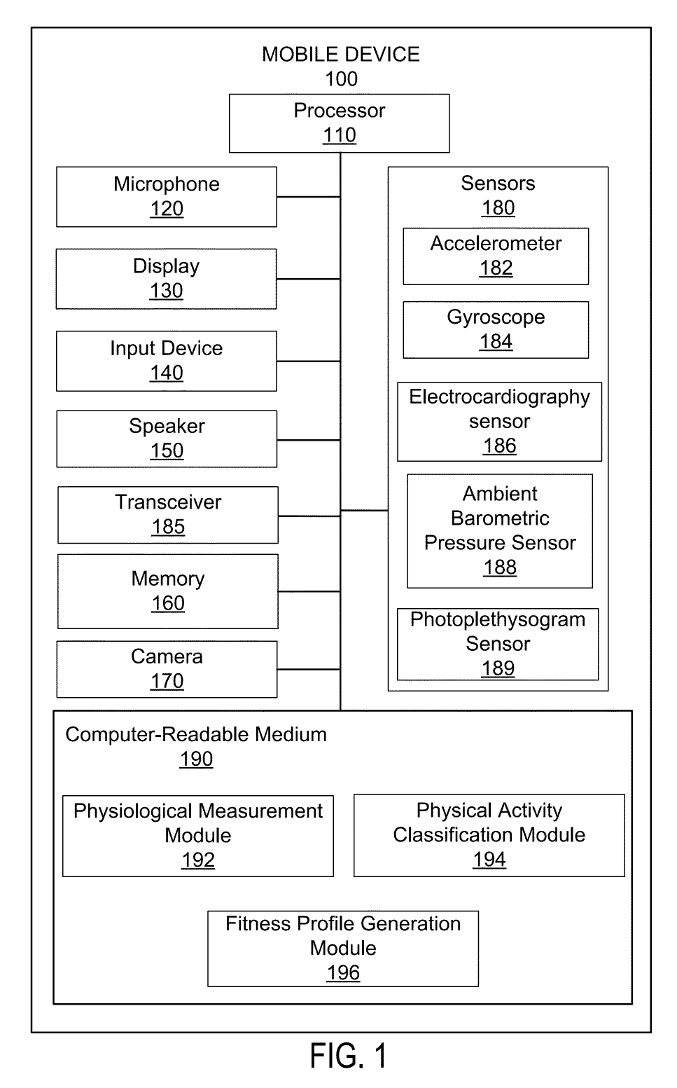

FIG. 1 is a block diagram of an exemplary mobile device 100, according to some implementations. Mobile device 100 includes a processor 110, microphone 120, display 130, input device 140, speaker 150, memory 160, camera 170, sensors 180, transceiver 185, and computer-readable medium 190.

Processor 110 may be any general-purpose processor operable to carry out instructions on the mobile device 100. The processor 110 is coupled to other units of the mobile device 100 including microphone 120, display 130, input device 140, speaker 150, memory 160, camera 170, sensors 180, transceiver 185, and computer-readable medium 190.

Microphone 120 may be any an acoustic-to-electric transducer or sensor that converts sound into an electrical signal. The microphone 120 may provide functionality for a user of the mobile device 100 to record audio or issue voice commands for the mobile device 100.

Display 130 may be any device that displays information to a user. Examples may include an LCD screen, CRT monitor, or seven-segment display.

Input device 140 may be any device that accepts input from a user. Examples may include a keyboard, keypad, or mouse. In some embodiments, the microphone 120 may also function as an input device 140.

Speaker 150 may be any device that outputs sound to a user. Examples may include a built-in speaker or any other device that produces sound in response to an electrical audio signal and/or ultrasonic signal(s).

Memory 160 may be any magnetic, electronic, or optical memory. It can be appreciated that memory 160 may include any number of memory modules. An example of memory 160 may be dynamic random access memory (DRAM).

Camera 170 is configured to capture one or more images via a lens located on the body of mobile device 100. The captured images may be still images or video images. The camera 170 may include a CMOS image sensor to capture the images. Various applications running on processor 110 may have access to camera 170 to capture images. It can be appreciated that camera 170 can continuously capture images without the images actually being stored within the mobile device 100. Captured images may also be referred to as image frames.

Sensors 180 may be a plurality of sensors configured to obtain data accessible by the processor. The sensors 180 may also be physically coupled to the outer body of the mobile device 100. The plurality of sensors 180 may include accelerometer 182, gyroscope 184, ambient barometric pressure sensor 188, PPG HR sensor 189, and/or electrocardiography sensor 186. The accelerometer may be configured to obtain data used to determine measure motion input from the mobile device 100. The gyroscope 184 may be configured to obtain data used to determine orientation of the mobile device 100. The ambient barometric pressure sensor 186 may be configured to obtain data used to determine altitude and change of thereof of the mobile device 100. Together, or individually, the measurements from the accelerometer 182, ambient pressure sensor 186 and gyroscope 184 can be used by the physical activity classification module 198 to classify a physical activity being performed by a user of the mobile device 100 (described below). The electrocardiography sensor 186 may be configured to obtain data used to determine a heart rate measurement of the user, via one or more electrodes on the outer body of the mobile device 100 that contact the user's skin. PPG HR sensor 189 may be configured to obtain data used to determine a HR measurement of the user via optical transduction. It can be appreciated that the sensors 180 can include any other sensors used to obtain data for facilitating measurements by the mobile device 100. The position sensor 188 may be configured to obtain a position measurement of the mobile device 100. The position sensor 188 may be a linear, angular, or multi-axis position sensor. In some embodiments, the position sensor 188 may be a GPS device.

Transceiver 185 may be configured to communicate from the mobile device 100 to a second device using any network protocol. The transceiver 185 may communicate via NFC, Bluetooth, Wi-Fi, etc.

Computer-readable medium 190 may be any magnetic, electronic, optical, or other computer-readable storage medium. Computer-readable medium 190 includes physiological measurement module 192, physical activity classification module 194, and fitness profile generation module 196.

Physiological measurement module 192, when executed by processor 110, can be configured to cause the mobile device 100 to determine one or more physiological measurements of the user. The one or more physiological measurements may be determined via data obtained by the sensors 180. For example, the electrocardiography sensor 186 may obtain data that can be used by physiological measurement module 192 to determine a heart rate of the user. The physiological measurement module 192 may also determine a transient physiological measurement from the determined physiological measurements. For example, the physiological measurement module 192 may determine a HRR measurement from a first determined heart rate measurement and a second determined heart rate measurement. Other physiological measurements can include, but is not limited to, an electromyography response measurement or a blood pressure measurement.

Physical activity classification module 194, when executed by processor 110, can be configured to cause the mobile device 100 to classify a physical activity being performed by the user. The physical activity can be classified via data obtained by the sensors 180. For example, the accelerometer 182 and/or gyroscope 184 may obtain motion input data that can be used by the physical activity classification module 194 to classify the physical activity being performed by the user. The motion input data from the accelerometer 182 and/or gyroscope 184 can be used collectively or individually by the physical activity classification module 194. For example, certain motion input data obtained by the accelerometer 182 and/or gyroscope 184 may allow for the physical activity classification module 194 to determine that the user is running, while other motion input data obtained by the accelerometer 182 and/or gyroscope 184 may allow for the physical activity classification module 194 to determine that the user is cycling. In another example, ambient barometric pressure sensor 186 can be used in conjunction with accelerometer 182 to determine if user gaining elevation (e.g., ascending descending the stairs), or loosing elevation (e.g. running downhill).

The fitness profile generation module 196, when executed by processor 110, can be configured to cause the mobile device 100 to generate a fitness profile for the user based on the determinations made by the physiological measurement module 192 and physical activity classification module 194, described above. The fitness profile generation module 196 may use the transient physiological measurement determined by the physiological measurement module 192, the classified physical activity determined by the physical activity classification module 194, and various other contextual information (e.g., location, time, date) to generate the fitness profile. More specifically, the fitness profile generation module 196 may correlate the transient physiological measurement determined by the physiological measurement module 192 to the classified physical activity determined by the physical activity classification module 194. The fitness profile may indicate the user's overall fitness state based on the aforementioned determined information. The generated fitness profile may be displayed to the user via display 130.

It can be appreciated that the outer body of the mobile device 100 may be sized to be portable for a user. It can be appreciated that the term “portable” may refer to something that is able to be easily carried or moved, and may be light and/or small. In the context of embodiments of the present invention, the term portable may refer to something easily transportable by the user or wearable by the user. For example, the mobile device 100 may be a smartphone device or a watch wearable by the user. Other examples of portable devices include a head-mounted display, calculator, portable media player, digital camera, pager, personal navigation device, etc. Examples of devices that may not be considered portable include a desktop computer, traditional telephone, television, appliances, etc. It can be appreciated that the physiological measurements can be obtained via the smartphone, watch, or any other of the mentioned devices.

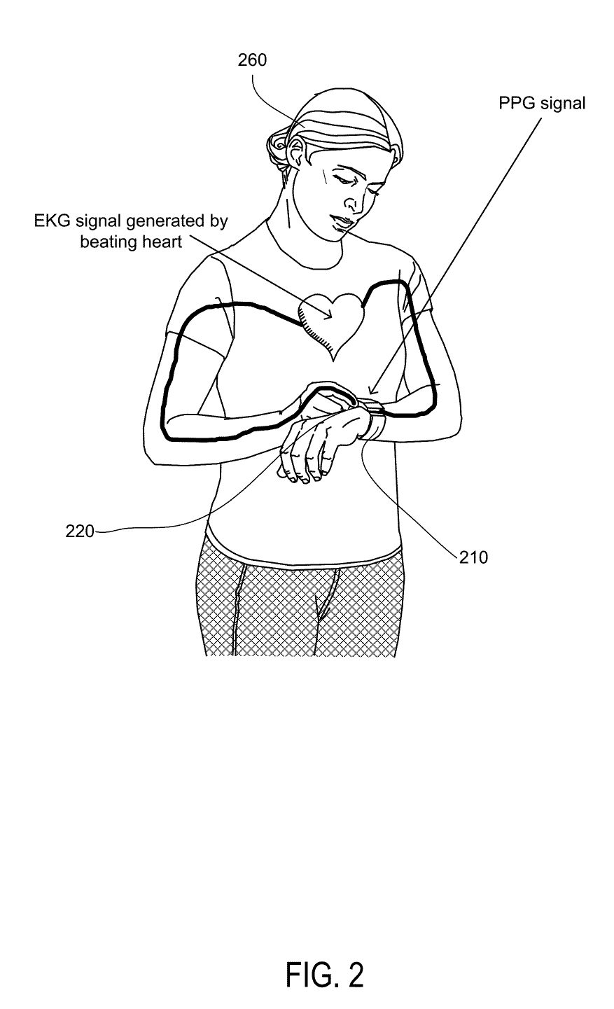

FIG. 2 illustrates a mobile device 210 configured to obtain bodily function measurements of a user, according to some implementations. In this particular example, the mobile device 210 may be a wristwatch device worn around the user's 260 wrist. However, it is understood that a wristwatch is just one example of a mobile device 210 that can be used in implementations described herein. The mobile device 210 may obtain EKG and/or PPG measurements of the user 260 which can be used to determine the user's heart rate during a physical activity. It can be appreciated that in addition to the EKG and/or PPG measurements, the mobile device 210 can obtain various other physiological measurements of the user 260. In some embodiments, one or more sensors may be placed at the bottom of the mobile device 210, where the contact makes a continuous contact with the user's 260 wrist while the user 260 wears the mobile device 210. In some implementations, the mobile device 210 may include a contact layer including, e.g., silver metal or Indium Tin Oxide (ITO).

The mobile device 210 may also include a multifunction button 220, which may be used to obtain a physiological measurement and also as a user input device. For example, the multifunction button 220 may be used by the user 260 to set a date and/or time for the mobile device 210. The multifunction button may have an integrated sensor (e.g., EKG sensor and/or PPG) on the surface. The user 260 may also use the multifunction button 220 to obtain an EKG measurement by touching the multifunction button 220 that has the integrated sensor. In some embodiments, the multifunction button 220 may be integrated into a touchscreen of the mobile device 210.

For example, when the user is wearing the mobile device 210, his/her wrist may be touching one or more of the sensors. As a result, the sensors may complete a circuit through the user's 260 body. The mobile device 210 may then measure an electrical potential through the completed circuit to determine the EKG measurement. Alternatively, and not illustrated in FIG. 2, sensors positioned and/or touched at other locations, for example legs, feet, ankles, knees, elbows, arms, neck, head, etc. could also be used to generate EKG and/or PPG, depending on the location and how the contact was made. It can be appreciated that the EKG and/or PPG measurement may be obtained opportunistically by the mobile device 210. That is, the EKG and/or PPG measurement may be obtained during the course of the user's regular activities without active participation on the part of the user. The mobile device 210 may regularly scan and store data needed for physiological measurements of the user 260 in the user's normal course of operating the mobile device 210, without the user wanting or needing a particular vital sign report at that time.

Additionally, the HR measurements may be obtained during the course of physical activity being performed by the user. Accordingly, by obtaining multiple HR measurements of the user 260, the user's heart rate at multiple times during the physical activity can be determined. From these heart rate determinations, the mobile device 210 can determine a heart rate recovery (HRR) measurement of the user (as described above with respect to FIG. 1). Additionally, as described above, various sensors within the mobile device 210 can be used to classify the physical activity being performed by the user. The HRR measurement can be correlated to the classified physical activity and a fitness profile can be generated for the user. The fitness profile can indicate the user's overall fitness state (described further below).

The mobile device 210 may be designed to be portable such that the user may easily wear the device or carry it on his/her person. In some embodiments, the mobile device 210 may perform everyday functions other than obtaining HR measurements of the user. For example, the mobile device 210 may provide the current time, a stopwatch function, a calendar function, communication functions, etc. The HR measurement functions may be available in addition to the other described functions on the wristwatch device.

In some implementations, the mobile device 210 may communicate with another mobile device or non-mobile device. For example, the wristwatch can communicate with a smartphone or desktop computer. The wristwatch may transmit the sensor data to the smartphone or desktop computer and the processing of the data may be carried out on the smartphone or desktop computer. The smartphone or desktop computer may then generate the fitness profile for the user based on the sensor data. In some implementations, the mobile device 210 can simply be one or more sensors without any other primary function. The sensors may then communicate the sensor data to another mobile device or non-mobile device.

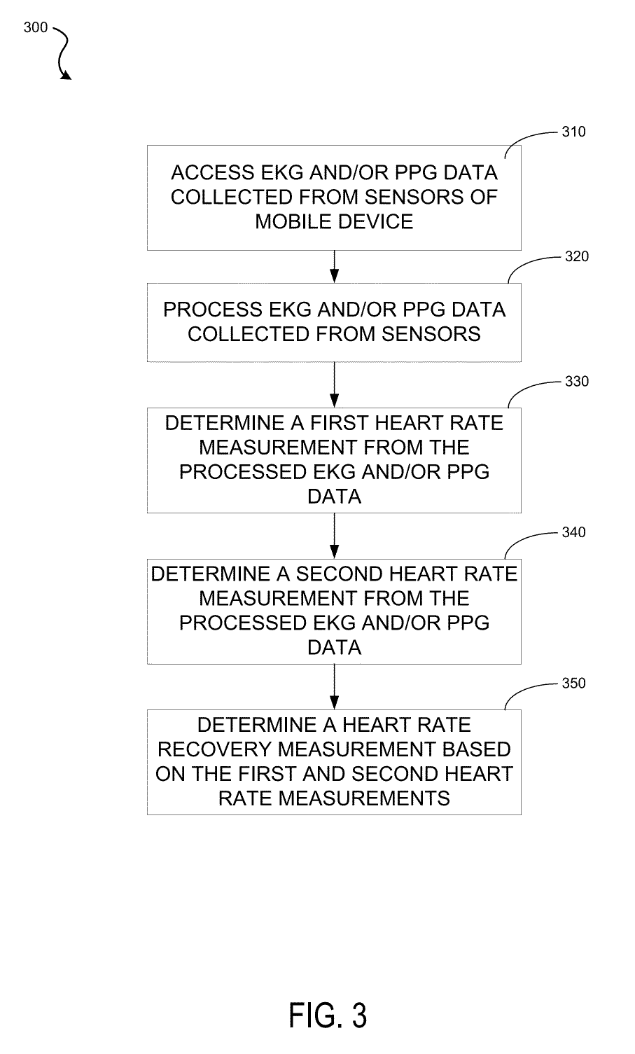

FIG. 3 is a flow diagram 300 of a process for determining a heart rate recovery (HRR) measurement of a user, according to some implementations. In block 310, HR data collected from sensors of the mobile device is accessed. The HR data may be accessed by the processor of the mobile device. The sensors may be located on the outer body of the mobile device. For example, in the case of a wristwatch, the sensors may be located beneath the face of the wristwatch or along the strap. The data may have been collected opportunistically by the sensors. That is, the sensors may have collected the data during the “regular” routine of the user, without any active participation on the part of the user.

In block 320, after the HR data collected from the sensors of the mobile device is accessed, the HR data collected from the sensors is processed. The data may be manipulated by the processor of the mobile device such that the data can provide meaningful information. That is, various algorithms may be applied to the EKG and/or PPG data to make sense of the EKG and/or PPG data such that a heart rate or other meaningful information can be determined from the processed data. For example, an R-peaks detection algorithm may be applied to the EKG data. Processing the HR data may also include arranging the data in a chronological order and associating specific data sets with specific timestamps.

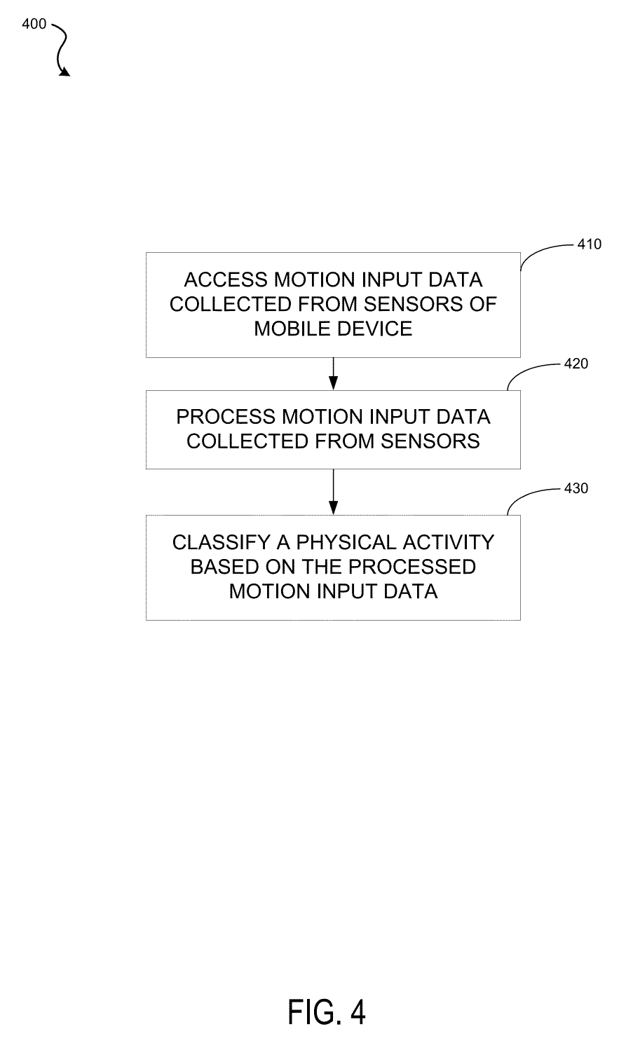

In block 330, after the HR data is collected from the sensors is processed, a first rate measurement is determined from the processed HR data. The first heart rate measurement may be determined based on HR data obtained while the user is performing a physical activity. More specifically, the first heart rate measurement may be determined based on HR data obtained while the user was performing the physical activity at an intense level. For example, the user may have been running, climbing a flight of stairs, or cycling at a fast pace. Indications that the user was performing a physical activity at the time may be inferred from data obtained by other sensors within the mobile device (e.g., accelerometer, gyroscope, etc.), which is described in further detail with respect to FIG. 4.

In block 340, after the first heart rate measurement is determined, a second heart rate measurement is determined from the processed HR data. The second heart rate measurement may be determined based on HR data obtained while the user is performing the same physical activity described above. More specifically, the second heart rate measurement may be determined based on HR data obtained while the user was performing the physical activity at a level above resting. For example, the user may have slowed from a running pace to a jogging pace, just finished climbing the flight of stairs, or may have slowed from cycling at a fast pace to cycling at a moderate pace.

In block 350, after the second heart rate measurement is determined, a heart rate recovery (HRR) measurement is determined based on the first and second heart rate measurements. HRR measurements are well known in the art and can be indicative of physical cardiac condition and the risk of certain diseases. In some implementations, the HRR measurement may be a measurement of the difference in the user's heart rate during the physical activity being performed at an intense level and the user's heart rate during the physical activity being performed at a level above resting, over time. It can be appreciated that typical HRR measurements may take the difference between heart rate during an intense activity and heart rate at resting, over time. However, in this implementation, many advantages can be realized by taking the difference between the heart rate at the intense level and the heart rate at the level above resting. For example, in many instances, a user may not transition directly from performing a physical activity to resting. Instead, it is more likely that the user may transition from an intense level of physical activity to a moderate level of physical activity before transitioning to resting.

FIG. 4 is a flow diagram 400 of a process for classifying a physical activity, according to some implementations. In block 410, motion input data collected from sensors within a mobile device is collected. The sensors may include, but is not limited to, an accelerometer, gyroscope, inclinometer, etc. The motion input data may be accessed by the processor of the mobile device. The sensors may be located within the mobile device, as shown with respect to FIG. 1. In some implementations, the motion input data may be collected by the sensors in an ongoing fashion. That is, the motion input data may be collected by the sensors throughout at all times while the user is in possession of the mobile device. In some implementations, the motion input data may be collected upon movement of the mobile device. That is, the motion input data may be collected when movement on the part of the user is detected. In some implementations, the sensors may collect and store sensor data even if the device is asleep.

In block 420, after the motion input data is accessed, the motion input data collected from the sensors is processed. The motion input data may be manipulated by the processor of the mobile device such that the data can provide meaningful information. That is, various algorithms may be applied to the motion input data to make sense of the motion input data such that a user movement can be classified or other meaningful information can be determined from the processed data. For example, the motion input data may indicate what type of movement the user is experiencing, such as driving, walking, running or sleeping. Processing the motion input data may also include arranging the data in a chronological order and associating specific data sets with specific timestamps.

In block 430, after the motion input data is processed, a physical activity is classified based on the processed motion input data. The physical activity can be classified within predefined categories of activities based on an inference made from the motion input data. For example, if the motion input data is indicative of the user running, the physical activity may be classified as “running” Moreover, the classification of the physical activity can include the intensity of the physical activity, which also can be inferred from the motion input data. For example, the physical activity may be classified as “intense running” or “jogging”, etc. Other examples of physical activity classifications can include, but is not limited to, walking, cycling, climbing stairs, stationary running, swimming, etc.

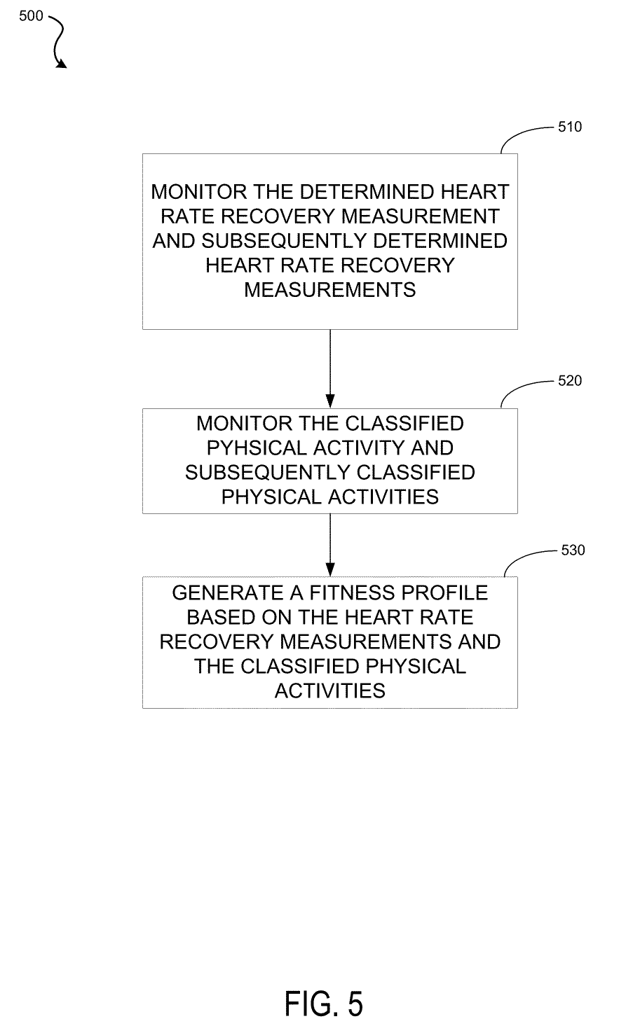

FIG. 5 is a flow diagram 500 of a process for generating a fitness profile for a user, according to some implementations. In block 510, the determined heart rate recovery measurement and subsequently determined heart rate recovery measurements may be monitored. The subsequently determined heart rate recovery measurements may have been determined using the process outlined with respect to FIG. 3. The heart rate recovery measurements may be stored within memory of the mobile device. The heart rate recovery measurements may have associated timestamp data and location data indicating when and where the measurements were taken.

In block 520, after the determined heart rate recovery measurement and subsequently determined heart rate recovery measurements are monitored, the classified physical activity and subsequently classified physical activities are monitored. The subsequently classified physical activities may have been determined using the processed outlined with respect to FIG. 4. The classifications of the physical activities may be stored within memory of the mobile device. The classifications of the physical activities may have associated timestamp data and location data indicating when and where the physical activity the user was participating was determined and classified.

It can be appreciated that the order of block 510 and block 520 may be interchangeable.

In block 530, after the classified physical activity and subsequently classified physical activities are monitored, a fitness profile is generated based on the heart rate recovery measurements and the classified physical activities. The fitness profile may provide an overview to the user of his/her overall fitness. Additionally, the fitness profile may indicate the user's fitness as it pertains to the different classified physical activities. The determined heart rate recovery measurements may be correlated to the classified physical activities and used in the generation of the fitness profile. The fitness profile may provide the HRR and classified physical activity statistics over specified period of time (e.g., one day, one week, one month, one year, etc.). In some implementations, specified period of time may be indicated by the user while in other implementations the specified period of time may be independently determined by the mobile device.

It can be appreciated that the generation of the fitness profile may run as a service on the mobile device that collects the HRR data and the physical activity classification data over time in order to provide an ongoing health predictor to the user. The classified physical activities and determined HRR measurements can be categorized by time, location, and activity type. Moreover, the user may be able to apply various filters to the generated fitness profile in order to view data that the user deems pertinent to himself/herself (e.g., cycling only, month of June, etc.).

The details of the fitness profiled are described in further detail below.

It can also be appreciated that while the embodiments described herein describe using an EKG measurement for obtaining the user's HR, any other type of measurement may be used in accordance with the systems and methods described herein. For example, photoplethysmogram, measurements, impedance plethysmography, etc.

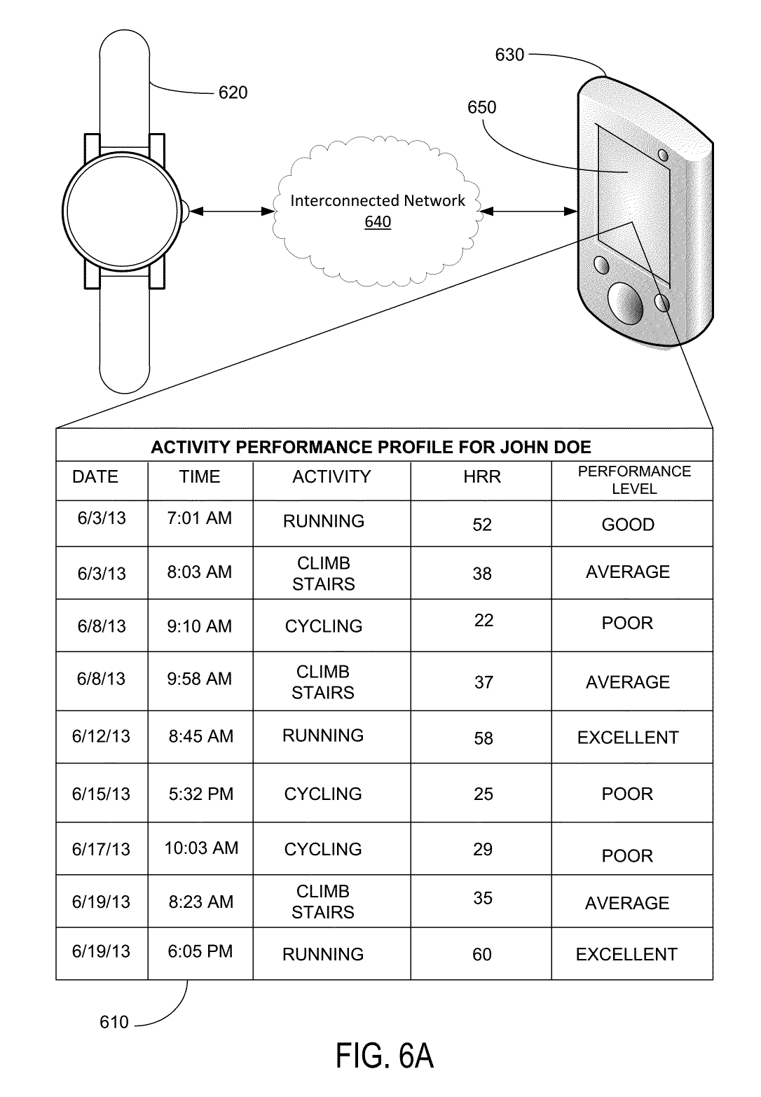

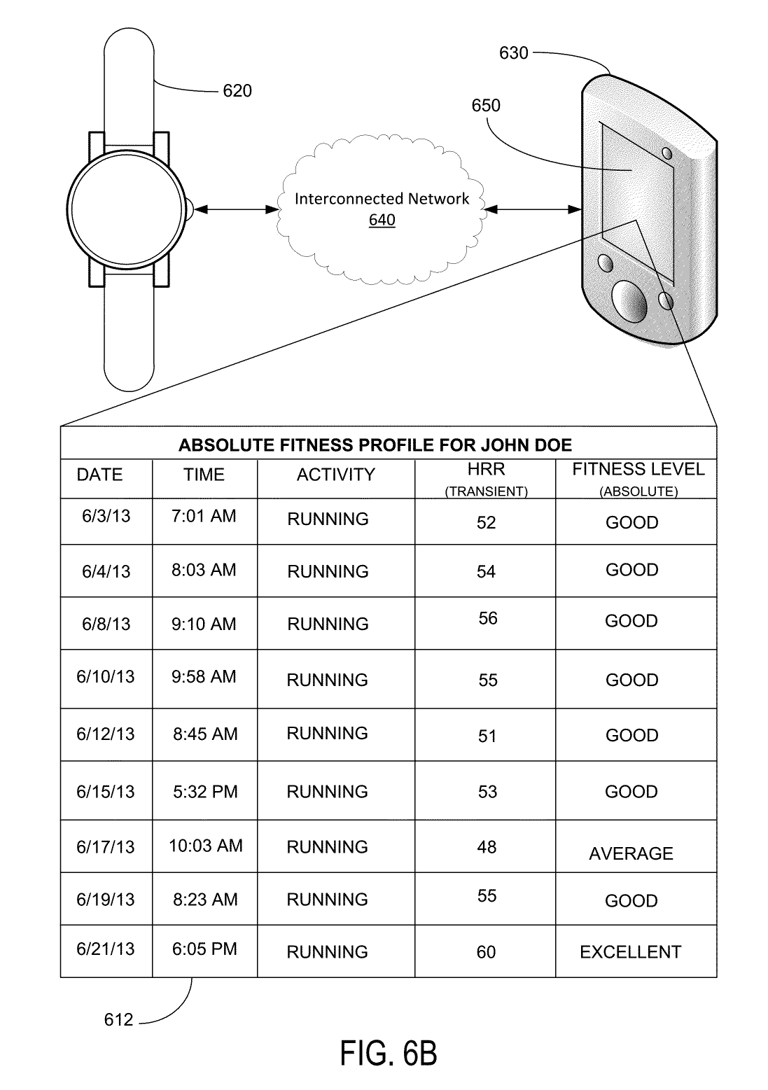

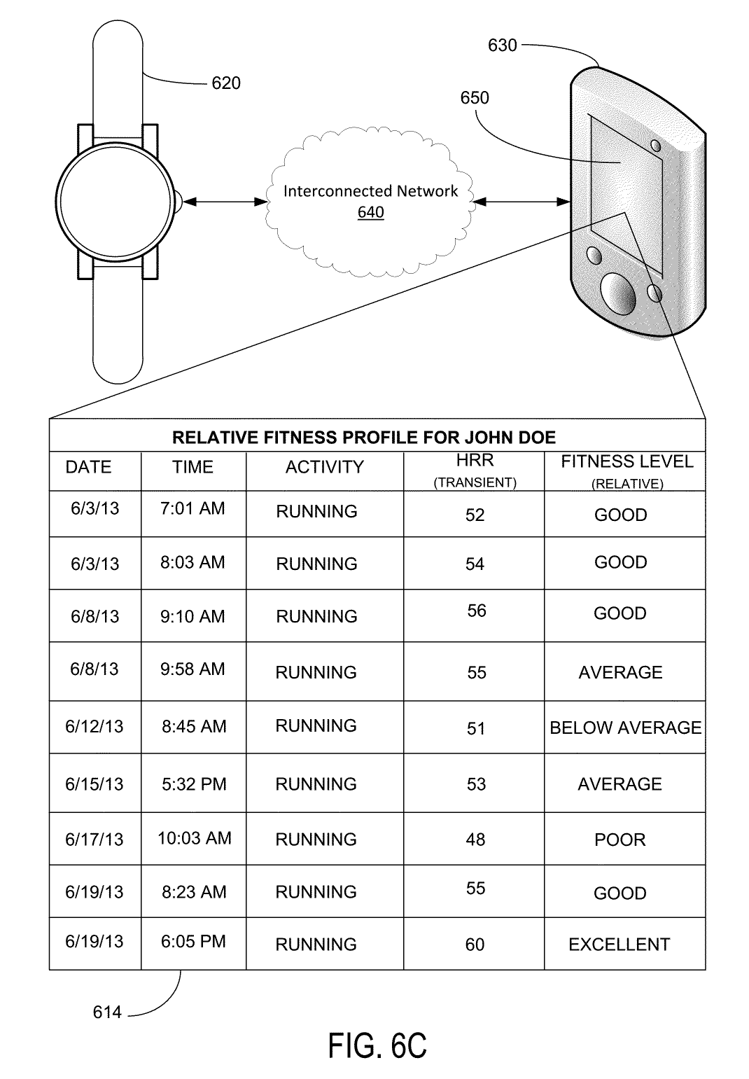

FIG. 6A illustrates an exemplary performance level profile 610 displayed on a mobile device. More specifically, FIG. 6A shows a first mobile device 620 and a second mobile device 630. It can be appreciated that either first mobile device 620 or second mobile device 630 can be implemented using elements of the mobile device in FIG. 1. The first mobile device 620 and the second mobile device 630 may communicate with each other via interconnected network 640. A few examples of interconnected network 640 include, but is not limited to, Bluetooth, Wi-Fi, the Internet, etc. In this example, the first mobile device 620 may be a wristwatch (e.g., smart-watch) and the second mobile device 630 may be a smartphone device.

The stored HRR measurements, physical activity classifications, and other pertinent data may be communicated to the second mobile device 630 from the first mobile device 620, via interconnected network 640. The data may be communicated in real-time as it is captured by the first mobile device 620, or may be communicated at pre-defined times or pre-defined time intervals. The user may then view the fitness profile on a display 650 of the second mobile device 630. In some implementations, the user may view the fitness profile directly on a display of the first mobile device 620, without any need for a second mobile device 630 or interconnected network 640.

As shown in FIG. 6A, the performance profile 610 is for user “John Doe.” As described above, the performance profile may be viewed on a display 650 of the second mobile device 630. The performance profile 610 depicts the classified physical activities that user John Doe participated in along with the correlated HRR measurement for each activity. Additionally, the performance profile provides a date and time at which the physical activity occurred and a fitness level indication. In this example, nine data points are depicted in the performance profile 610, however it can be appreciated that any number of data points may be shown as part of the performance profile 610. Further, the user may apply filters based on date, activity, etc. to increase or decrease the number data points shown in the performance profile 610. In some implementations, the performance profile 610 may also include a location at which the user performed the physical activities.

As can be seen in FIG. 6A, user John Doe has participated in three different physical activities between Jun. 3, 2013 and Jun. 19, 2013. These physical activities include running, climbing stairs, and cycling. In this example, the user climbs stairs in the morning and it may be inferred by the physical activity classification module 194 (FIG. 1) that this may be a regular routine that the user performs (e.g., perhaps on the way to work every morning). Similarly, the physical activity classification module 194 may also make inferences with regards to the running activity and the cycling activity. For each classified physical activity, a correlated HRR measurement is shown. The correlated HRR measurement may be the difference between the user's measured heart rate at an intense or elevated activity level during the physical activity and the user's measured heart rate at a moderate or decreased activity level during the physical activity. It can be appreciated that the moderate or decreased activity level may be a level above a resting activity level when the user is at rest.

The performance profile 610 indicates that the user is an excellent runner and in very good shape for running activities. The user's HRR for running activities varies from 52-60, which may be considered between good to excellent. The HRR may indicate the difference between the user's measured heart rate while he may have been running vigorously and when the user may have been jogging, over a predefined period of time (e.g., one minute, two minutes, etc.). The performance profile 610 also indicates that the user has average fitness for climbing stairs and poor fitness for cycling. The “overall picture” provided for John Due by the performance profile 610 is that he is an excellent runner, average at everyday activities such as climbing stairs, and a poor cyclist. This may indicate to user John Doe that he should partake in more physical conditioning to improve his fitness for cycling.

It can be appreciated that while the first mobile device 620 my capture data pertaining to physical activity and heart rate measurements while the user is resting, this data may not be shown in the performance profile 610 as the performance profile 610 aims to correlate HRR measurements between intense activity levels and moderate activity levels instead of between intense activity levels and resting activity levels. The performance profile 610 may also indicate the duration of the classified physical activity.

In some implementations, the fitness profile 610 may also present a graph to the user. The graph may plot the various data points currently shown in the fitness profile 610. The graph may also show historical averages, trends, etc. to provide an overall fitness level of the user. The performance profile may also identify which activities the user commonly engages in and can provide a fitness profile exclusively for the specific physical activity. The heart rate recovery measurements may be averaged over a certain time period to provide the user with an average fitness level for the classified physical activity, or a combination of all of the classified physical activities.

FIG. 6B illustrates an exemplary absolute fitness profile 612 displayed on a mobile device 630. The absolute fitness profile 612 indicates the user's absolute fitness based on the transient HRR measurement for the recorded physical activity, the user's age, the user's gender, etc. As can be seen in FIG. 6B, user John Doe has participated in nine running activities between Jun. 3, 2014 and Jun. 21, 2013. The running activities may have been classified by the physical activity classification module 194, as described above. For each running activity, a correlated HRR measurement is shown. The absolute fitness profile 612 indicates the user's absolute fitness for, in this example, running. Based on each HRR transient measurement, a correlated measurement for John Doe's fitness level for running is provided. For example, on days where John Doe's running activities resulted in a HRR transient measurement between 50-59, a determination was made that John Doe's absolute fitness level for running is “GOOD”. On days where John Doe's running activities resulted in a HRR transient measurement between 40-49, a determination was made that John Doe's absolute fitness level for running is “AVERAGE”. On days where John Doe's running activities resulted in a HRR transient measurement above 60, a determination was made that John Doe's absolute fitness level for running is “EXCELLENT”. Other exemplary absolute fitness level measurements can include “BELOW AVERAGE” for HRR transient measurements 30-39 and below and “POOR” for HRR transient measurements below 29. In some embodiments, the absolute fitness level measurements may be different for varying activities, varying genders, and varying ages of users.

FIG. 6C illustrates an exemplary relative fitness profile displayed on a mobile device 630. The relative fitness profile 614 indicates the user's relative fitness level for a particular activity against previously measured fitness levels, to provide the user with some indication of how their fitness level is improving/declining over time. As can be seen in FIG. 6C, user John Doe has participated in nine running activities between Jun. 3, 2014 and Jun. 21, 2013. The running activities may have been classified by the physical activity classification module 194, as described above. For each running activity, a correlated HRR measurement is shown. The relative fitness profile 614 indicates the user's relative fitness for, in this example, running. Comparing the observed HRR measurement for completed activity to one or more previous HRR transient measurements for previously completed activities, a correlated measurement for John Doe's relative fitness is provided. For example, John Doe's relative fitness measurement can be “EXCELLENT”, “GOOD”, “AVERAGE”, “BELOW AVERAGE”, or “POOR” depending on the HRR transient measurement of the completed activity as compared to one or more previous HRR measurements observed for previously completed activities. On some days, John Doe may have had poorer activity performance due to some external factor (e.g., sickness, etc.). While on other days, John Doe may have improved performance over previously completed activities. The relative fitness profile 614 provides John Doe a picture of his fitness level over time.

It can be appreciated that while the disclosure described herein discusses heart rate transients based on attenuation of the heart rate from an increased level to a decreased level, all of the implementations described can also apply to heart rate transients based on ramping up of the heart rate from a decreased level to an increased level.

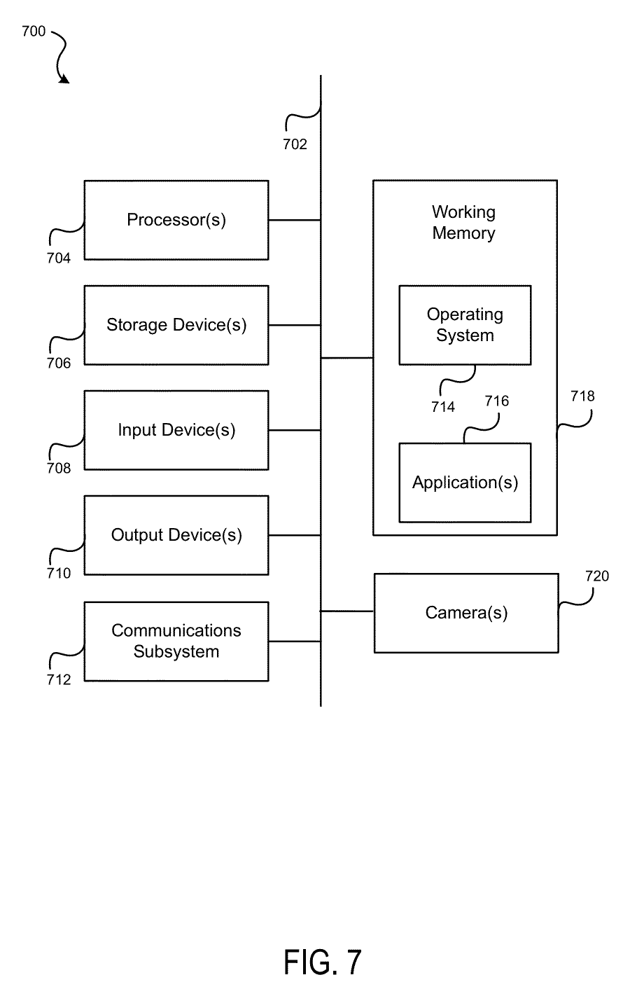

FIG. 7 illustrates an example of a computing system in which one or more embodiments may be implemented. A computer system as illustrated in FIG. 7 may be incorporated as part of the above described computerized device. For example, computer system 700 can represent some of the components of a television, a computing device, a server, a desktop, a workstation, a control or interaction system in an automobile, a tablet, a netbook or any other suitable computing system. A computing device may be any computing device with an image capture device or input sensory unit and a user output device. An image capture device or input sensory unit may be a camera device. A user output device may be a display unit. Examples of a computing device include but are not limited to video game consoles, tablets, smart phones and any other hand-held devices. FIG. 7 provides a schematic illustration of one embodiment of a computer system 700 that can perform the methods provided by various other embodiments, as described herein, and/or can function as the host computer system, a remote kiosk/terminal, a point-of-sale device, a telephonic or navigation or multimedia interface in an automobile, a computing device, a set-top box, a table computer and/or a computer system. FIG. 7 is meant only to provide a generalized illustration of various components, any or all of which may be utilized as appropriate. FIG. 7, therefore, broadly illustrates how individual system elements may be implemented in a relatively separated or relatively more integrated manner. In some embodiments, elements of computer system 700 may be used to implement functionality of the mobile device 100 in FIG. 1.

The computer system 700 is shown comprising hardware elements that can be electrically coupled via a bus 702 (or may otherwise be in communication, as appropriate). The hardware elements may include one or more processors 704, including without limitation one or more general-purpose processors and/or one or more special-purpose processors (such as digital signal processing chips, graphics acceleration processors, and/or the like); one or more input devices 708, which can include without limitation one or more cameras, sensors, a mouse, a keyboard, a microphone configured to detect ultrasound or other sounds, and/or the like; and one or more output devices 710, which can include without limitation a display unit such as the device used in embodiments of the invention, a printer and/or the like.

In some implementations of the embodiments of the invention, various input devices 708 and output devices 710 may be embedded into interfaces such as display devices, tables, floors, walls, and window screens. Furthermore, input devices 708 and output devices 710 coupled to the processors may form multi-dimensional tracking systems.

The computer system 700 may further include (and/or be in communication with) one or more non-transitory storage devices 706, which can comprise, without limitation, local and/or network accessible storage, and/or can include, without limitation, a disk drive, a drive array, an optical storage device, a solid-state storage device such as a random access memory (“RAM”) and/or a read-only memory (“ROM”), which can be programmable, flash-updateable and/or the like. Such storage devices may be configured to implement any appropriate data storage, including without limitation, various file systems, database structures, and/or the like.

The computer system 700 might also include a communications subsystem 712, which can include without limitation a modem, a network card (wireless or wired), an infrared communication device, a wireless communication device and/or chipset (such as a Bluetooth™ device, an 802.11 device, a Wi-Fi device, a WiMax device, cellular communication facilities, etc.), and/or the like. The communications subsystem 712 may permit data to be exchanged with a network, other computer systems, and/or any other devices described herein. In many embodiments, the computer system 700 will further comprise a non-transitory working memory 718, which can include a RAM or ROM device, as described above.

The computer system 700 also can comprise software elements, shown as being currently located within the working memory 718, including an operating system 714, device drivers, executable libraries, and/or other code, such as one or more application programs 716, which may comprise computer programs provided by various embodiments, and/or may be designed to implement methods, and/or configure systems, provided by other embodiments, as described herein. Merely by way of example, one or more procedures described with respect to the method(s) discussed above might be implemented as code and/or instructions executable by a computer (and/or a processor within a computer); in an aspect, then, such code and/or instructions can be used to configure and/or adapt a general purpose computer (or other device) to perform one or more operations in accordance with the described methods.

A set of these instructions and/or code might be stored on a computer-readable storage medium, such as the storage device(s) 706 described above. In some cases, the storage medium might be incorporated within a computer system, such as computer system 700. In other embodiments, the storage medium might be separate from a computer system (e.g., a removable medium, such as a compact disc), and/or provided in an installation package, such that the storage medium can be used to program, configure and/or adapt a general purpose computer with the instructions/code stored thereon. These instructions might take the form of executable code, which is executable by the computer system 700 and/or might take the form of source and/or installable code, which, upon compilation and/or installation on the computer system 700 (e.g., using any of a variety of generally available compilers, installation programs, compression/decompression utilities, etc.) then takes the form of executable code.

Substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed. In some embodiments, one or more elements of the computer system 700 may be omitted or may be implemented separate from the illustrated system. For example, the processor 704 and/or other elements may be implemented separate from the input device 708. In one embodiment, the processor is configured to receive images from one or more cameras that are separately implemented. In some embodiments, elements in addition to those illustrated in FIG. 7 may be included in the computer system 700.

Some embodiments may employ a computer system (such as the computer system 700) to perform methods in accordance with the disclosure. For example, some or all of the procedures of the described methods may be performed by the computer system 700 in response to processor 704 executing one or more sequences of one or more instructions (which might be incorporated into the operating system 714 and/or other code, such as an application program 716) contained in the working memory 718. Such instructions may be read into the working memory 718 from another computer-readable medium, such as one or more of the storage device(s) 706. Merely by way of example, execution of the sequences of instructions contained in the working memory 718 might cause the processor(s) 704 to perform one or more procedures of the methods described herein.

The terms “machine-readable medium” and “computer-readable medium,” as used herein, refer to any medium that participates in providing data that causes a machine to operate in a specific fashion. In some embodiments implemented using the computer system 700, various computer-readable media might be involved in providing instructions/code to processor(s) 704 for execution and/or might be used to store and/or carry such instructions/code (e.g., as signals). In many implementations, a computer-readable medium is a physical and/or tangible storage medium. Such a medium may take many forms, including but not limited to, non-volatile media, volatile media, and transmission media. Non-volatile media include, for example, optical and/or magnetic disks, such as the storage device(s) 706. Volatile media include, without limitation, dynamic memory, such as the working memory 718. Transmission media include, without limitation, coaxial cables, copper wire and fiber optics, including the wires that comprise the bus 702, as well as the various components of the communications subsystem 712 (and/or the media by which the communications subsystem 712 provides communication with other devices). Hence, transmission media can also take the form of waves (including without limitation radio, acoustic and/or light waves, such as those generated during radio-wave and infrared data communications).

Common forms of physical and/or tangible computer-readable media include, for example, a floppy disk, a flexible disk, hard disk, magnetic tape, or any other magnetic medium, a CD-ROM, any other optical medium, punchcards, papertape, any other physical medium with patterns of holes, a RAM, a PROM, EPROM, a FLASH-EPROM, any other memory chip or cartridge, a carrier wave as described hereinafter, or any other medium from which a computer can read instructions and/or code.

Various forms of computer-readable media may be involved in carrying one or more sequences of one or more instructions to the processor(s) 704 for execution. Merely by way of example, the instructions may initially be carried on a magnetic disk and/or optical disc of a remote computer. A remote computer might load the instructions into its dynamic memory and send the instructions as signals over a transmission medium to be received and/or executed by the computer system 700. These signals, which might be in the form of electromagnetic signals, acoustic signals, optical signals and/or the like, are all examples of carrier waves on which instructions can be encoded, in accordance with various embodiments of the invention.

The communications subsystem 712 (and/or components thereof) generally will receive the signals, and the bus 702 then might carry the signals (and/or the data, instructions, etc. carried by the signals) to the working memory 718, from which the processor(s) 704 retrieves and executes the instructions. The instructions received by the working memory 718 may optionally be stored on a non-transitory storage device 1006 either before or after execution by the processor(s) 704.

The methods, systems, and devices discussed above are examples. Various configurations may omit, substitute, or add various procedures or components as appropriate. For instance, in alternative configurations, the methods may be performed in an order different from that described, and/or various stages may be added, omitted, and/or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Also, configurations may be described as a process which is depicted as a flow diagram or block diagram. Although each may describe the operations as a sequential process, many of the operations can be performed in parallel or concurrently. In addition, the order of the operations may be rearranged. A process may have additional steps not included in the figure. Furthermore, examples of the methods may be implemented by hardware, software, firmware, middleware, microcode, hardware description languages, or any combination thereof. When implemented in software, firmware, middleware, or microcode, the program code or code segments to perform the necessary tasks may be stored in a non-transitory computer-readable medium such as a storage medium. Processors may perform the described tasks.

Having described several example configurations, various modifications, alternative constructions, and equivalents may be used without departing from the spirit of the disclosure. For example, the above elements may be components of a larger system, wherein other rules may take precedence over or otherwise modify the application of the invention. Also, a number of steps may be undertaken before, during, or after the above elements are considered.

What is claimed is:

1. A method for assessing a fitness state of a user via a mobile device, comprising:
obtaining, via one or more sensors, a first physiological measurement of the user during a first level of a physical activity;
obtaining, via the one or more sensors, a second physiological measurement during a second level of the physical activity;
determining, via a processor of the mobile device, a transient physiological measurement based on the first physiological measurement and the second physiological measurement;
classifying the physical activity based on one or more motion measurements obtained via the one or more sensors;
generating a fitness profile indicative of the fitness state of the user based at least in part on the determined transient physiological measurement and the classified physical activity.

2. The method of claim 1, wherein the first physiological measurement and the second physiological measurement comprises at least one of a heart rate measurement, an electromyography response measurement, a photoplethysmogram measurement, an impedance plethysmography measurement, or a blood pressure measurement.

3. The method of claim 1, wherein the determined transient physiological measurement comprises a heart rate recovery (HRR) measurement.

4. The method of claim 1, wherein the one or more sensors comprises at least an accelerometer.

5. The method of claim 1, further comprising monitoring the determined transient physiological measurement and subsequently determined transient physiological measurements over a period of time.

6. The method of claim 5, wherein generating the fitness profile is further based at least in part on subsequently classified physical activities and the subsequently determined transient physiological measurements.

7. The method of claim 1, wherein generating the fitness profile is further based at least in part on a frequency of the user to engage in the classified physical activity.

8. The method of claim 1, further comprising presenting, via a display of the mobile device, the fitness profile to the user within a graphical user interface (GUI).

9. The method of claim 1, wherein classifying the physical activity comprises categorizing the physical activity into one or more predefined physical activity categories.

10. The method of claim 1, wherein the first level of the physical activity and the second level of the physical activity are above a level of physical activity indicative of the user being in a resting state.

11. The method of claim 1, wherein the first physiological measurement and the second physiological measurement are opportunistically obtained by the mobile device.

12. A mobile device for assessing a fitness state of a user, comprising:
an outer body sized to be portable for the user;
a processor contained within the outer body;
a plurality of sensors coupled to the outer body for obtaining data accessible by the processor;
wherein one or more of the sensors is configured to obtain a first physiological measurement of the user during a first level of a physical activity;
wherein one or more of the sensors is configured to obtain a second physiological measurement of the user during a second level of the physical activity; and
wherein the processor is configured to:
determine a transient physiological measurement based on the first physiological measurement and the second physiological measurement;
classify the physical activity based on one or more motion measurements obtained via the one or more sensors; and
generate a fitness profile indicative of the fitness state of the user based at least in part on the determined transient physiological measurement and the classified physical activity.

13. The mobile device of claim 12, wherein the first physiological measurement and the second physiological measurement comprises at least one of a heart rate measurement, an electromyography response measurement, a photoplethysmogram measurement, an impedance plethysmography measurement, or a blood pressure measurement.

14. The mobile device of claim 12, wherein the determined transient physiological measurement comprises a heart rate recovery (HRR) measurement.

15. The mobile device of claim 12, wherein the one or more sensors comprises at least an accelerometer.

16. The mobile device of claim 12, wherein the processor is further configured to monitor the determined transient physiological measurement and subsequently determined transient physiological measurements over a period of time.

17. The mobile device of claim 16, wherein generating the fitness profile is further based at least in part on subsequently classified physical activities and the subsequently determined transient physiological measurements.

18. The mobile device of claim 12, wherein generating the fitness profile is further based at least in part on a frequency of the user to engage in the classified physical activity.

19. The mobile device of claim 12, further comprising a display configured to present the fitness profile to the user within a graphical user interface (GUI).

20. The mobile device of claim 12, wherein classifying the physical activity comprises categorizing the physical activity into one or more predefined physical activity categories.

21. The mobile device of claim 12, wherein the first level of the physical activity and the second level of the physical activity are above a level of physical activity indicative of the user being in a resting state.

22. The mobile device of claim 12, wherein the first physiological measurement and the second physiological measurement are opportunistically obtained by the mobile device.

23. An apparatus for assessing a fitness state of a user, comprising:
means for obtaining, via one or more sensors, a first physiological measurement of the user during a first level of a physical activity;
means for obtaining a second physiological measurement during a second level of the physical activity;
means for determining, via the one or more sensors, a transient physiological measurement based on the first physiological measurement and the second physiological measurement;
means for classifying the physical activity based on one or more motion measurements obtained via the one or more sensors;
means for generating a fitness profile indicative of the fitness state of the user based at least in part on the determined transient physiological measurement and the classified physical activity.

24. The apparatus of claim 23, further comprising means for monitoring the determined transient physiological measurement and subsequently determined transient physiological measurements over a period of time.

25. The apparatus of claim 23, wherein the first level of the physical activity and the second level of the physical activity are above a level of physical activity indicative of the user being in a resting state.

26. The apparatus of claim 23, wherein the first physiological measurement and the second physiological measurement are opportunistically obtained.

27. One or more non-transitory computer-readable media storing computer-executable instructions for assessing a fitness state of a user that, when executed, cause one or more computing devices included in a mobile device to:
obtain, via one or more sensors, a first physiological measurement of the user during a first level of a physical activity;
obtain, via the one or more sensors, a second physiological measurement during a second level of the physical activity;
determine, via a processor of the mobile device, a transient physiological measurement based on the first physiological measurement and the second physiological measurement;
classify the physical activity based on one or more motion measurements obtained via the one or more sensors;
generate a fitness profile indicative of a fitness state of the user based at least in part on the determined transient physiological measurement and the classified physical activity.

28. The non-transitory computer-readable media of claim 27, further comprising means for monitoring the determined transient physiological measurement and subsequently determined transient physiological measurements over a period of time.

29. The non-transitory computer-readable media of claim 27, wherein the first level of the physical activity and the second level of the physical activity are above a level of physical activity indicative of the user being in a resting state.

30. The non-transitory computer-readable media of claim 27, wherein the first physiological measurement and the second physiological measurement are opportunistically obtained.
